(19) United States
(12) Patent Application Publication
(10) Pub. No.: US 2017/0258604 A1
(43) Pub. Date: Sep. 14, 2017

(54) INTERVERTEBRAL PLATE SYSTEM

(71) Applicant: Spinal USA, Inc., Parsippany, NJ (US)

(72) Inventors: Patrick Barrett, Jackson, MS (US); James Milton Phillips, Star, MS (US)

(21) Appl. No.: 15/299,175

(22) Filed: Oct. 20, 2016

Related U.S. Application Data
(62) Division of application No. 12/883,130, filed on Sep. 15, 2010, now abandoned.

(51) Int. Cl. A61F 2/44; A61F 2/46; A61B 17/70; A61B 17/86; A61F 2/28; A61F 2/30

(52) U.S. Cl. CPC A61F 2/4455; A61F 2/30744; A61B 17/7059; A61F 2002/30433; A61F 2002/30448; A61F 2002/305; A61F 2002/30509; A61F 2002/30774; A61F 2002/30785; A61F 2002/30787; A61B 17/86; A61F 2002/2835; A61F 2002/30451; A61F 2/4611

(57) ABSTRACT

Devices and methods are provided for assisting in spinal stabilization. An improved intervertebral plate system is provided that includes an intervertebral spacer, a curvilinear plate, a plurality of bone screws, a curvilinear cover element and a cover screw. The curvilinear plate is configured and arranged to at least inhibit the intervertebral spacer from backing out when positioned between the two vertebrae of a patient. The plate can be secured to one or more intervertebral bodies via a plurality of bone screws. The curvilinear cover element, which can have a smooth and uniform surface, can be attached to the plate. The cover element is configured to inhibit the plurality of bone screws from inadvertently backing out of the plate. The plate and/or the cover element can be substantially recessed within the intervertebral space, thereby reducing the risk of damage to tissue.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application is a divisional application of U.S. patent application Ser. No. 12/883,130, filed Sep. 15, 2010, the entire disclosure of which is hereby expressly incorporated by reference.

### BACKGROUND OF THE INVENTION

Field of the Invention

The present application relates to devices, systems and processes for spinal surgeries. In particular, the present application relates to devices, systems and processes for disc replacement surgeries.

Description of the Related Art

The spine relies on intervertebral spinal discs in between adjacent vertebrae to serve as mechanical cushions and transmit compressive loads. Spinal discs are composed of an outer annulus fibrosus that surrounds an inner nucleus pulposus. The annulus fibrosus is composed of laminae of fibrous tissue and fibrocartilage, while the nucleus pulposus is composed of water, chondrocytes, collagen fibrils and proteoglycan aggrecans that have hyaluronic long chains. The nucleus pulposus functions to distribute hydraulic pressure in all directions within each disc under compressive loads.

The nucleus pulposus, which begins early in life as eighty percent water, slowly desiccates with age. This causes the spinal disc to lose its cushioning ability and ability to bear loads, resulting in pain in the back and lower extremities. To resolve these problems, the degenerated nucleus may be removed and replaced. In some other cases, the nucleus may be removed and the vertebrae may be fused together in a spinal fusion procedure, which may include implanting an intervertebral cage and/or bone growth material to facilitate fusion of the vertebrae.

During vertebral disc replacement surgery, it is commonplace to insert an intervertebral spacer between two adjacent vertebrae in the place of a ruptured or diseased disc. Such intervertebral spacers can include, but are not limited to, bone grafts, peek cages, titanium cages, stainless steel cages, bioresorbable cages, and the like. In some circumstances, following implantation, these intervertebral spacers can inadvertently back out or be displaced from an intervertebral space. To prevent the intervertebral spacers from backing out, vertebral plates can be provided. While these vertebral plates prevent back out of intervertebral spacers, they are often located outside of the vertebrae, which can cause damage to adjacent blood vessels and even death of the patient. Further, individual components of the vertebral plates, such as screws inserted therein, can also become inadvertently loose and back out, thereby causing damage to adjacent blood vessels.

There remains a need for an intervertebral plate system that can retain an intervertebral spacer in situ which does not suffer from the deficiencies of conventional plates.

### SUMMARY OF SOME EMBODIMENTS

Devices and methods are provided for assisting in spinal stabilization. In some embodiments, a spinal implant system is provided. The system comprises an intervertebral spacer configured and arranged to be positioned between two vertebrae of a patient. The system further comprises a curvilinear plate configured and arranged to at least inhibit the intervertebral spacer from backing out when positioned between the two vertebrae of a patient. The curvilinear plate comprises sidewalls, a plurality of screw holes, a central screw hole for receiving a cover screw, and cover element contact surfaces. The system further comprises a plurality of bone screws adapted for insertion through the screw holes of the curvilinear plate and configured for anchoring the plate between two vertebrae of a patient. Also included in the system is a curvilinear cover element configured to inhibit the plurality of bone screws from backing out of the plate. The cover element is formed of an upper member and a lower member, the upper member having a smooth upper surface, the lower member having smooth lower surfaces for contacting the cover element contact surfaces of the plate, and a cover hole formed therethrough. The system further comprises a cover screw configured to be inserted through the cover hole to secure the cover element and the plate. The plate and the cover element are configured and arranged to be substantially recessed between two vertebrae when implanted.

In some embodiments, a spinal implant system is provided that comprises a plate configured and arranged to at least inhibit an intervertebral spacer from backing out when positioned between the two vertebrae of a patient, wherein the plate comprises a plurality of screw holes for receiving bone screws. The system further comprises a cover element configured to inhibit a plurality of bone screws from backing out of the plate when implanted in a patient, wherein the cover element has a surface that covers a substantial portion of a surface of the plate, and wherein the plate and cover element are arranged to be substantially recessed between two vertebrae of a patient when implanted.

In some embodiments, a spinal implant method is provided that comprises positioning an intervertebral spacer in an intervertebral space between two vertebrae of a patient; positioning a plate at least partially in said intervertebral space and adjacent to the spacer to at least inhibit the intervertebral spacer from backing out from said space; positioning a plurality of bone screws through the plate to anchor the plate between two vertebrae of a patient; and positioning a cover element at least partially in said intervertebral space and over the plate to inhibit the plurality of bone screws from backing out of the plate, wherein the cover element has a surface that covers a substantial portion of an upper surface of the plate. In addition, the plate and cover element are substantially recessed between two vertebrae of a patient when implanted such that they do not extend significantly beyond the outer surfaces of the two vertebrae.

### BRIEF DESCRIPTION OF THE DRAWINGS

FIGS. 1A and 1B illustrate components of an improved intervertebral plate system according to one embodiment of the present application.

FIGS. 2A and 2B illustrate different views of a plate according to one embodiment of the present application.

FIGS. 3A-3C illustrate different views of a cover element according to one embodiment of the present application.

FIG. 4 illustrates a cover screw according to one embodiment of the present application.

FIGS. 5A-5D illustrate a method of installing an improved intervertebral plate system according to one embodiment of the present application.

FIG. 6 illustrates a side view of an improved intervertebral plate system recessed in an intervertebral space according to one embodiment of the present application.

FIGS. 7A and 7B illustrate different views of an installation block according to one embodiment of the present application.

FIG. 8 illustrates an insertion tool according to one embodiment of the present application.

### DETAILED DESCRIPTION OF EMBODIMENTS

The present application relates to devices, systems and processes for spinal surgeries. In particular, the present application relates to an improved intervertebral plate system that can be used in disc replacement surgeries.

In some embodiments, the improved intervertebral plate system comprises an intervertebral spacer, a plate, and a cover element. The intervertebral spacer can be configured and arranged to be positioned or recessed between two vertebrae of a patient. Likewise, the plate can be securely positioned or recessed between the vertebrae, and can be configured and arranged to at least inhibit the intervertebral spacer from backing out when positioned between the two vertebrae. One or more bone screws can be provided through the plate and into adjacent vertebrae to assist in securing the plate to the vertebrae. The cover element can be securely fixed to the plate and can be configured to substantial cover the plate and inhibit the one or more bone screws from backing out of the plate. In some embodiments, the plate and cover element are substantially recessed between the two vertebrae of a patient such that very little, if any, of either component extends beyond the exposed surfaces of the vertebral bodies. Providing a plate and cover element that are substantially recessed between the two vertebrae advantageously prevents incidental damage caused by exposing the plate and/or cover element to blood vessels and tissue within the body.

One skilled in the art will appreciate that each of the components described above, including the intervertebral spacer, plate and cover element, has its own advantageous features, as discussed further below, such that they are not limited to use solely in combination with each other. For example, a system can comprise the intervertebral spacer and plate without the cover element, and can still provide numerous advantages over conventional plate systems.

Intervertebral Plate System

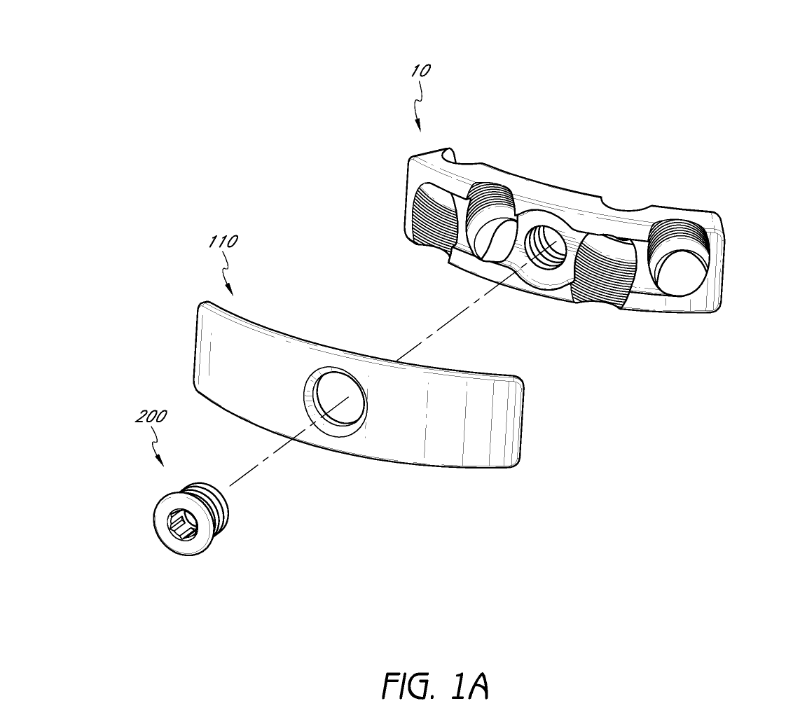

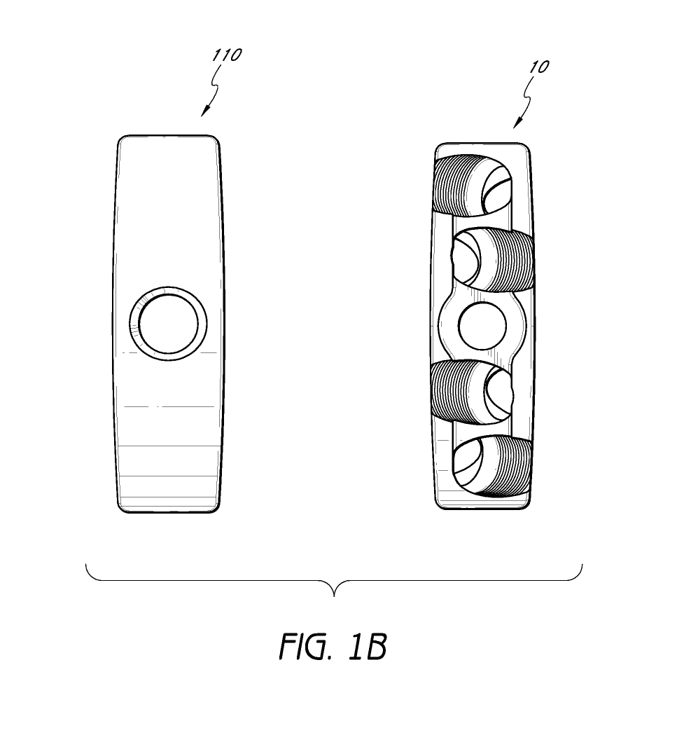

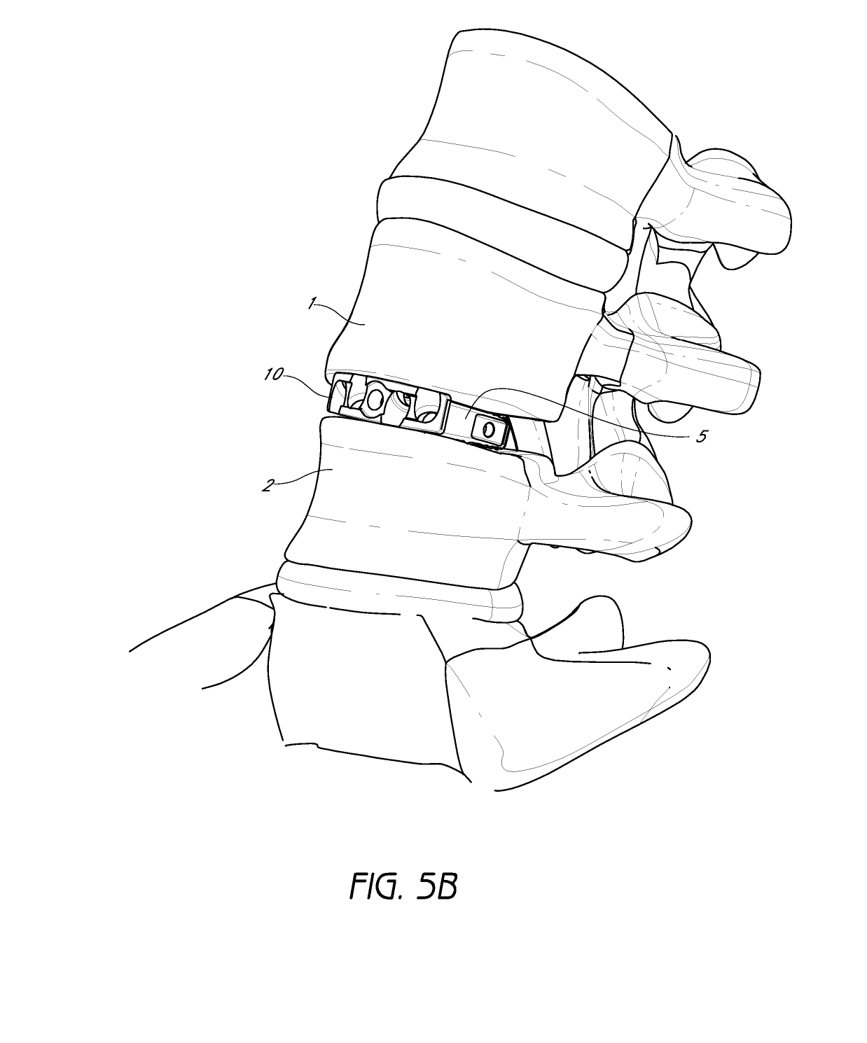

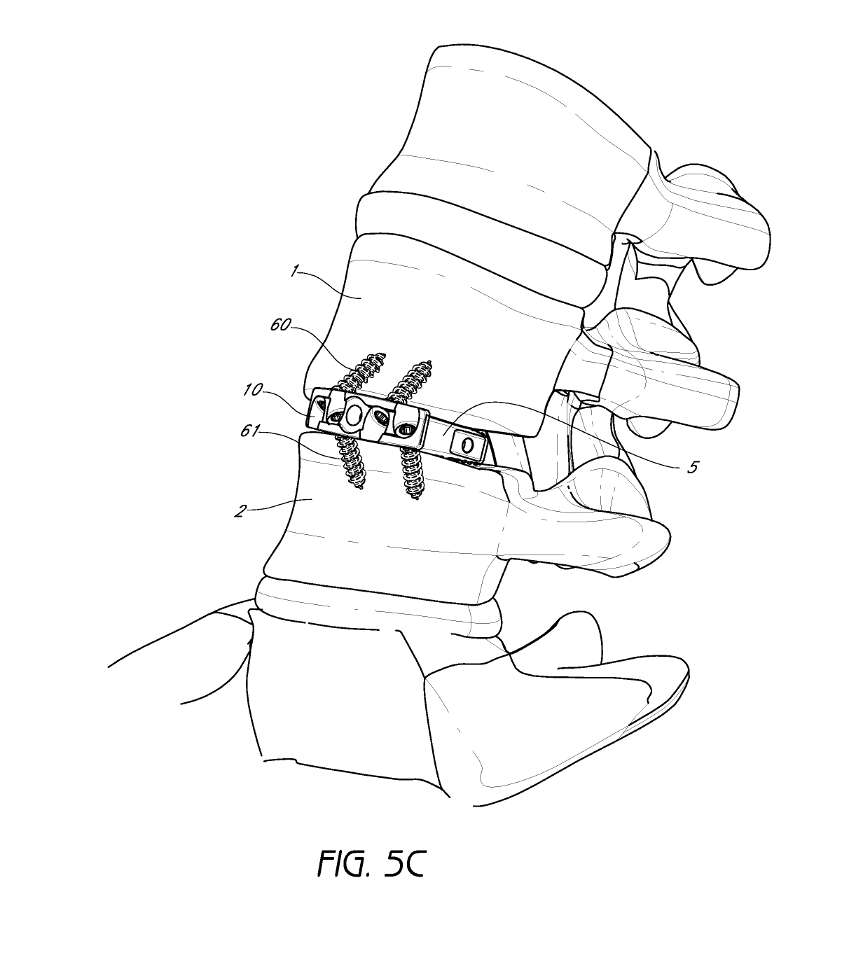

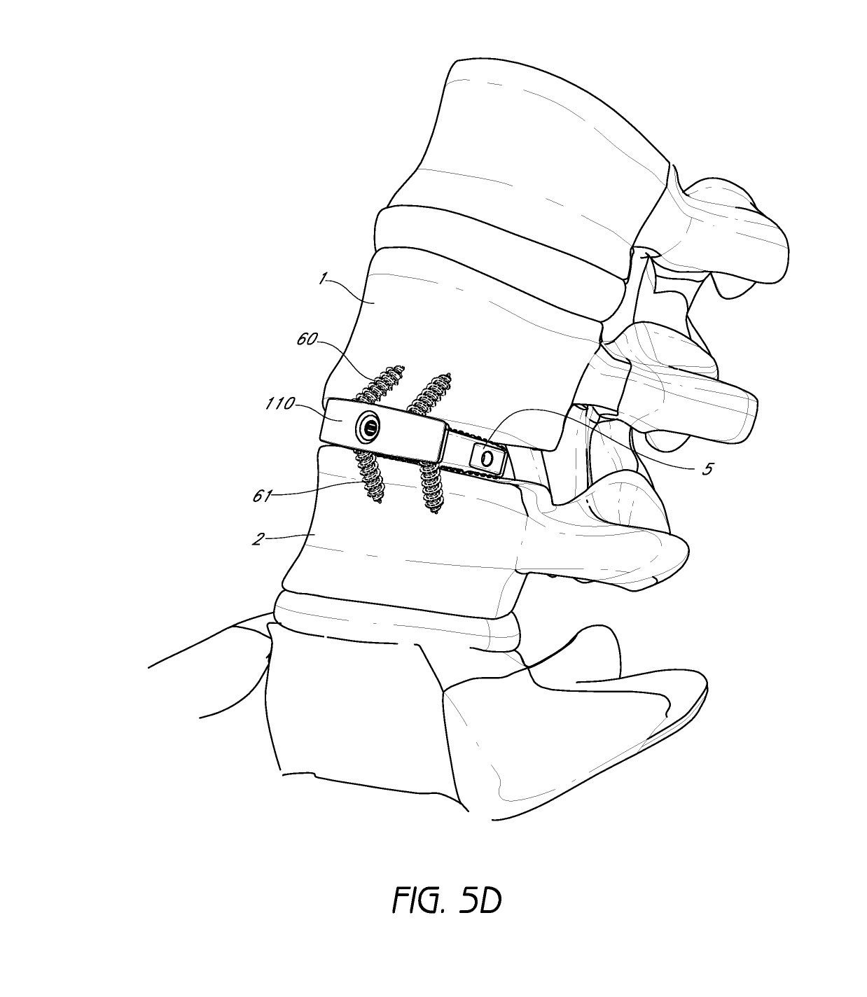

FIGS. 1A and 1B illustrate components of an improved intervertebral plate system according to one embodiment of the present application. The separate components include a plate 10, cover element 110 and cover screw 200. An intervertebral spacer, which can also be considered part of the system in some embodiments, is shown in FIG. 5A. In some embodiments, the plate 10, cover element 110 and cover screw 200 are used together with an intervertebral spacer. After implanting an intervertebral spacer, the plate 10 can be placed adjacent the intervertebral spacer to prevent or inhibit inadvertent backing out of the spacer from an intervertebral space. The cover element 110 can be placed adjacent the plate 10 to prevent or inhibit inadvertent backing out of the plate 10 itself, or screws within the plate, from an intervertebral space. The cover screw 200 can be used to secure the cover element 110 to the plate 10. For purposes of this application, the term “intervertebral space” can be any space in between adjacent vertebral bodies.

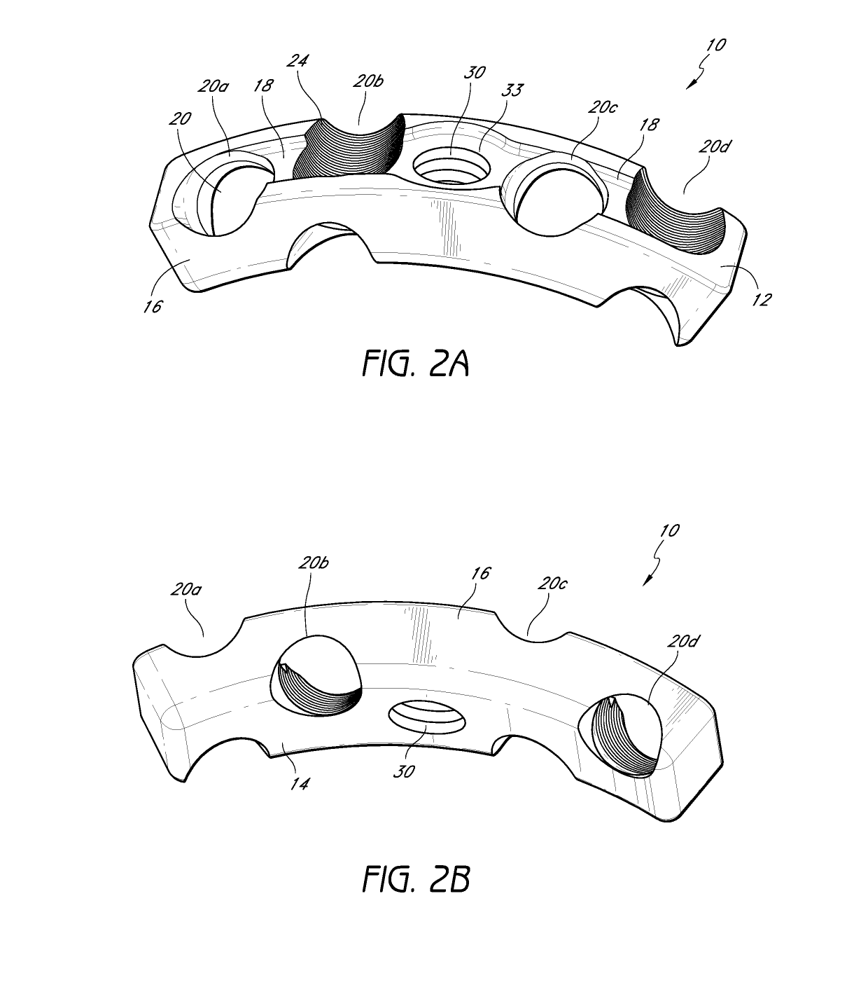

FIGS. 2A and 2B illustrate different views of a curvilinear plate according to one embodiment of the present application. The plate 10 includes a plate upper surface 12, a plate lower surface 14, and plate sidewalls 16. The plate 10 also includes a plurality of screw holes 20a, 20b, 20c and 20d, and a central screw hole 30 that are machined through the plate upper surface 12 and plate lower surface 14.

In some embodiments, the plate 10 will be positioned within an intervertebral space such that each of its sidewalls 16 are adjacent and/or in contact with first and second vertebral bodies, as shown in FIG. 5B. In this position, the lower surface 14 will face an intervertebral spacer, while the upper surface 12 will face outward from the intervertebral space. The plate 10 is advantageously sized and shaped such that it can be substantially or completely recessed within an intervertebral space between two vertebrae, such that little if any of the plate 10 is exposed outside of the intervertebral space. By being recessed within an intervertebral space, this advantageously reduces the contact between the plate 10 and tissue members near and surrounding the spine, thereby minimizing the risk of damage to such tissue.

As shown in FIGS. 2A and 2B, screw holes 20 are formed within the body of the plate 10. Each screw hole is designated as either screw hole 20a, 20b, 20c or 20d. In some embodiments, the screw holes are formed both within the upper surface 12 or lower surface 14, as well as the sidewall 16 of the plate 10. For example, FIG. 2A shows screw holes 20a and 20c formed within the upper surface 12 and plate sidewall 16. By having screw holes 20 that are formed within the plate sidewalls 16, this advantageously allows screws to be inserted at a desired angle when the plate is recessed in an intervertebral space.

The screw holes 20 can include interior threads 24 (shown in FIG. 2A) and are configured to receive one or more screws that can be secured to one or more vertebral bodies. Each of the screw holes 20 includes a longitudinal axis. In some embodiments, the longitudinal axis of one screw hole can be parallel to the longitudinal axis of another screw hole, while in other embodiments, the longitudinal axis of one screw hole can be non-parallel to the longitudinal axis of another screw hole, as illustrated in FIG. 2A. For example, in some embodiments, the longitudinal axis of screw hole 20a can be oriented at an angle of between 20 and 90 degrees, or between 45 and 90 degrees, away from the longitudinal axis of screw hole 20b. This results in screw hole 20a having a different orientation from screw hole 20b, and accordingly, a screw that is inserted into screw hole 20a will have a different orientation and alignment from a screw that is inserted into screw hole 20b.

By having two or more holes 20 with different longitudinal axes, this advantageously allows screws to be inserted through the plate 10 at a range of angles into various positions relative to one or more vertebral bodies, thereby allowing for a very stable placement of the plate 10 within an intervertebral space. For example, when the plate 10 is located in between two vertebrae, screw hole 20a can be configured such that an inserted screw 60 will be angled upwardly into contact with a first vertebral body 1, while screw hole 20b can be configured such that an inserted screw 61 will be angled downwardly into contact with a second vertebral body 2, as shown in FIG. 5C.

In the illustrated embodiments, the plate 10 includes two screw holes 20 having non-parallel longitudinal axes on each side of a central screw hole 30. This alternating configuration, in which screw hole 20a has a non-parallel longitudinal axis from screw hole 20b, which has a non-parallel longitudinal axis form screw hole 20c, which has a non-parallel longitudinal axis from screw hole 20d, advantageously allows the screws to secure the plate 10 to adjacent vertebral bodies at various angles, thereby providing improved security between the plate and vertebral bodies relative to conventional systems. In the illustrated embodiment, each of the screw holes 20a, 20b, 20c, and 20d has a longitudinal axis that is non-parallel to the others. In other embodiments, screw holes 20a and 20c share a parallel longitudinal axis, while screw holes 20b and 20d share a different, parallel longitudinal axis. One skilled in the art will appreciate that the plate 10 need not be limited to the illustrated configuration. For example, an alternative configuration provides for two screw holes 20 having parallel longitudinal axes on one side of a central screw hole 30, and two screw holes 20 having parallel longitudinal axes on the other side of the central screw hole 30. Moreover, while the illustrated embodiments include a total of four holes 20a, 20b, 20c and 20d in addition to the central screw hole 30, one skilled in the art will appreciate that more (e.g., five, six, seven, eight or more) or less holes (e.g., two or three) can be machined into the plate 10, thereby allowing a greater or lesser number of screws to be inserted into the vertebral bodies.

As shown in FIGS. 2A and 2B, a central screw hole 30 can be provided through the plate 10. The central screw hole 30 can include a threaded interior, and can be configured to receive a cover screw 200 that passes through a cover element 110, thereby securely fitting the cover element 110 to the plate 10. In some embodiments, the central screw hole 30 is of similar size and shape to any of the screw holes 20, while in other embodiments, the central screw hole 30 is of different size and shape from all of the screw holes 20. In alternative embodiments not shown, the screw hole for coupling the plate and cover element need not be centrally located. In some embodiments, there may not be a coupling screw hole at all; rather, the cover element can be secured to the plate member via other means, such as a snap fit.

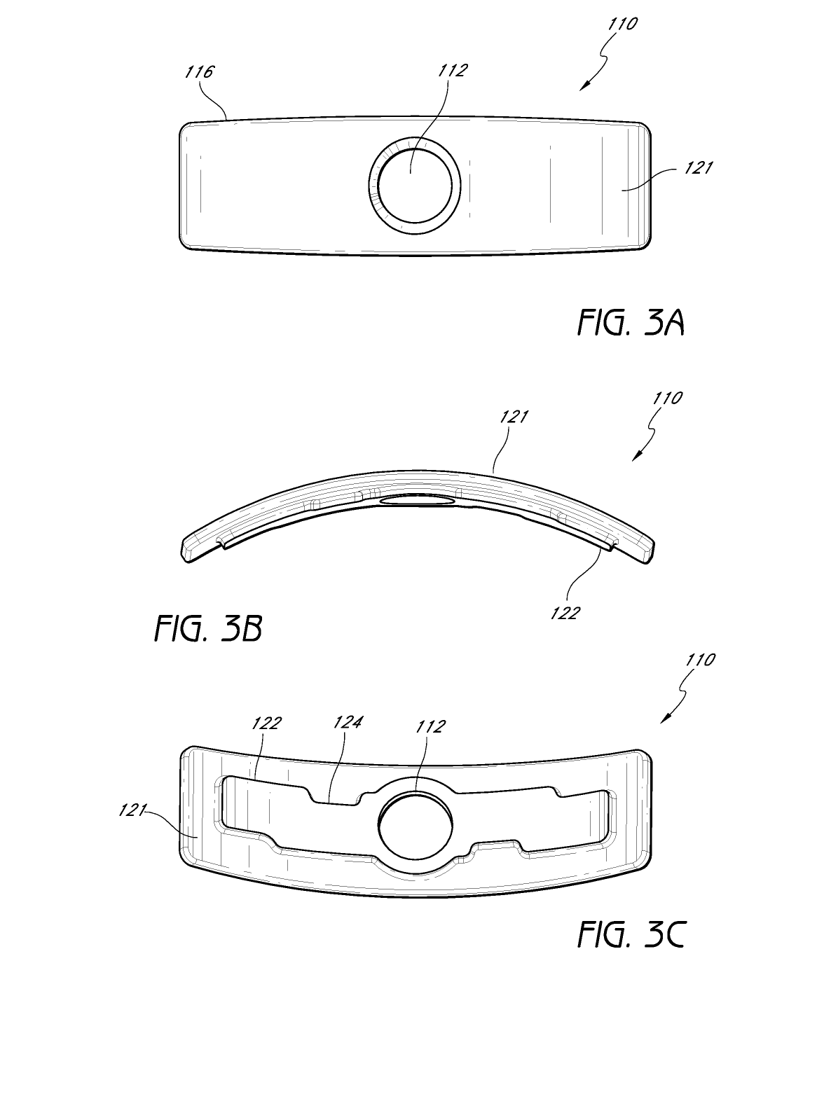

On the upper surface 12 of the plate 10, in between the central screw hole 30 and screw holes 20a and 20b, are contact surfaces 18. These contact surfaces 18 are designed to make contact with a lower member 122 of the cover element 110 (shown in FIG. 3B and discussed below). Providing a lower member 122 of the cover element 110 that is capable of contacting the contact surfaces 18 of the plate within the upper surface 12 of the plate 10 advantageously allows the cover element 110 to be oriented properly and securely fit with the plate 10 prior to securing the cover element 110 to the plate 10. Moreover, in some embodiments, the contact surfaces 18 can be slightly rough, thereby providing additional securing frictional forces between the plate 10 and cover element 110.

FIGS. 3A-3C illustrate different views of a cover element according to one embodiment of the present application. The cover element 110 includes an upper member and a lower member 122 as shown in FIG. 3B. The upper member 121 of the cover plate 110 includes a smooth upper surface 116, while the lower member 122 of the cover plate 110 includes a smooth lower surface 124. In the center of the cover element 110 is a cover hole 112.

In some embodiments, the cover element 110 can be coupled to the plate 10, such as via a cover screw 200 that is inserted through the cover hole 112 and the central screw hole 30. The cover element 110 can be provided after the plate 10 is securely positioned within an intervertebral space, such as by inserting screws through the screw holes 20 of the plate 10 and into adjacent vertebrae. Advantageously, the cover element 110 is sized and shaped such that it covers a substantial portion of the upper surface 12 of the plate 10, thereby preventing the inserted screws in screw holes 20 from unintentionally backing out. In some embodiments, the cover element covers a majority of the upper surface of the plate, and in some embodiments, the cover element covers most of the upper surface of the plate. In some embodiments, the tops of the bone screws inserted in the plate are substantially or completely covered by the cover plate. Moreover, the cover element 110 is sized and shaped such that it too can be substantially or completely recessed in between two vertebrae with the plate 10. By being substantially recessed in between two vertebrae, this reduces the exposure of the cover element 110 beyond the intervertebral space, which advantageously minimizes the risk of inadvertent contact between the cover element 110 and tissue, which could lead to tissue damage. In addition, the upper surface 116 of the cover element 110 is substantially smooth, thereby further reducing the risk of injury to tissue that may contact the cover element 110.

The cover element 110 includes an upper member 121 that is fixed to a lower member 122. In some embodiments, the upper member 121 and lower member 122 are two separate components that are fixed to each other, such as via a screw, adhesive, welding technique or any other machining process. In other embodiments, the upper member 121 and lower member 122 are formed from a monolithic piece. In some embodiments, both the upper member 121 and lower member 122 of the cover element 110 are curved to have a curvature substantially similar to that of the plate 10, thereby helping to facilitate coupling of the two components.

As shown in FIG. 3A, the upper member 121 of the cover element 110 has a substantially smooth upper surface 116. In some embodiments, the upper member 121 of the cover element can be substantially uniform such that it provides a continuous surface that helps to cover a substantial portion of the plate when the cover element and plate are coupled. This advantageously reduces the risk of damage to tissue that may come into contact with the cover element 110. In some embodiments, the cover element 110 will be substantially recessed in an intervertebral space along with the plate 10.

As shown in FIG. 3C, the lower member 122 of the cover element 110 is sized and shaped to fit within the contours of the upper surface 12 of the plate 10. The lower member 122 includes a smooth lower surface 124 that is configured to contact the contact surfaces 18 of the plate 10, thereby helping to properly align the cover element 110 and the plate 10.

The cover hole 112 is formed in the center of the cover element 110, and is configured to rest above the central screw hole 30 of the plate 10. A cover screw 200 can be inserted through the cover element 110 and plate 10, thereby securing the cover element 110 to the plate 10.

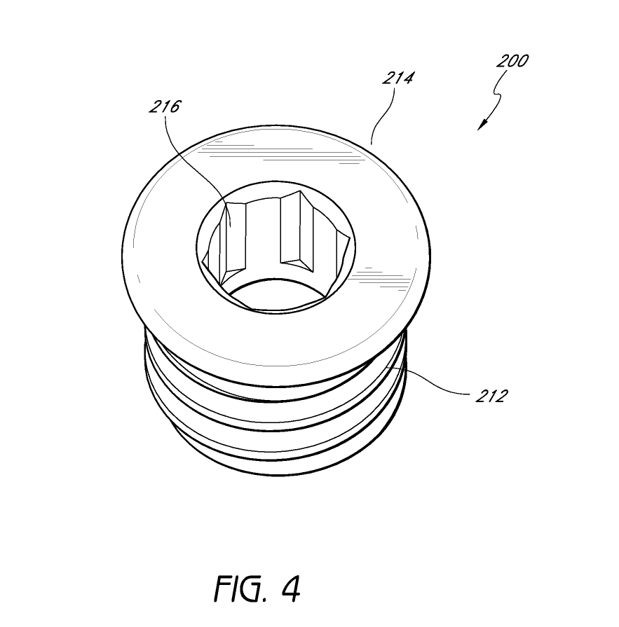

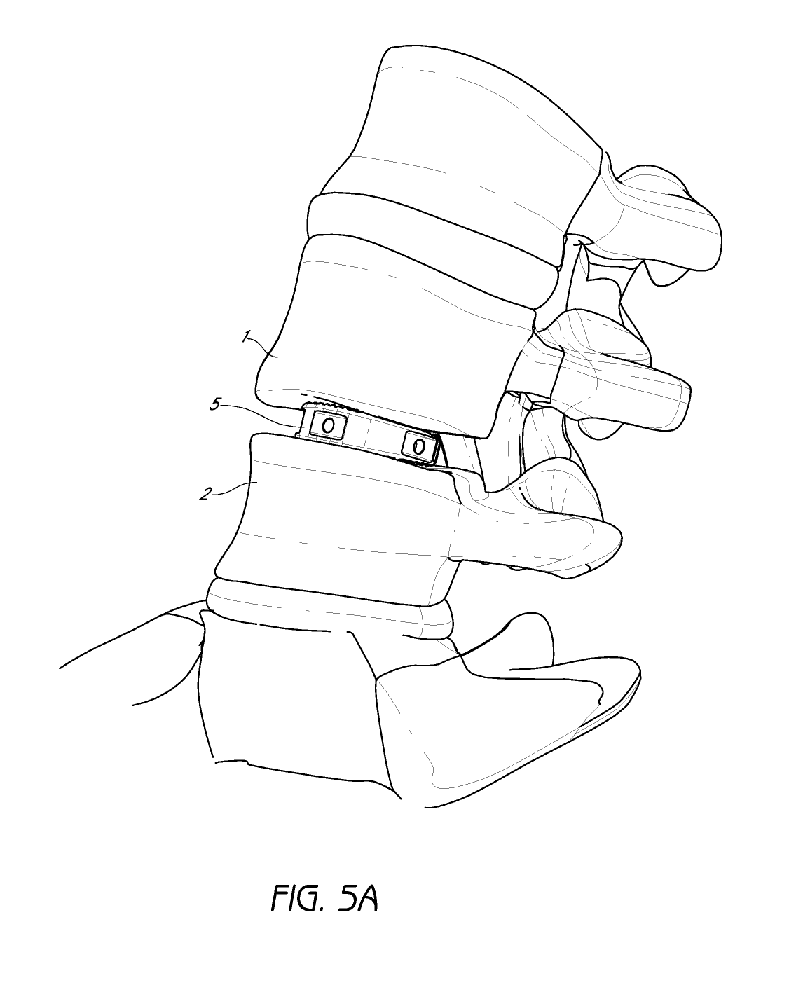

FIG. 4 illustrates a cover screw for securing the cover element 110 to the plate 10 according to one embodiment of the present application. The cover screw 200 includes a threaded portion 212 and head 214 with driving portion 216. An instrument, such as a screw driver, can be inserted into the driving portion 216 to rotate and insert the cover screw 200 into the cover element 110 and plate 10. When the cover screw 200 is completely threaded into the cover element 110 and plate 10, the head 207 of the cover screw 200 is positioned within the body of the cover element 110. This advantageously helps to reduce the risk of damage caused by tissue inadvertently contacting the cover screw 200.

### Methods of Using the Intervertebral Plate System

A procedure for using the improved intervertebral plate system according to one embodiment of the present application will now be described with respect to FIGS. 5A-5D. The procedure makes use of an intervertebral spacer 5, a plate 10, screws 60 and 61, and cover element 110.

First, an intervertebral spacer 5 is inserted and positioned into a disc space between a first vertebral body 1 (e.g., upper vertebral body) and a second vertebral body 2 (e.g., lower vertebral body), as shown in FIG. 5A. The intervertebral spacer 5 can include bone grafts or cages that can be positioned and fixed within an intervertebral space. In some embodiments, the intervertebral spacer 5 can be attached to first and/or second vertebral bodies. In some embodiments, the intervertebral spacer 5 can include VertiFuse™ Bone Grafts or the ALIF Peek Cages, produced and marketed by Spinal USA, LLC. The intervertebral spacer 5 can be recessed within the disc space such that it is not positioned beyond the exposed outer surfaces of vertebral body 1 or vertebral body 2. The intervertebral spacer 5 can serve to replace in whole or in a part a portion of the natural disc. As shown herein, an anterior approach may be used in some embodiments. In other embodiments other approaches may be used. For example, a lateral approach, a posterior approach, and/or a posterior-lateral approach could be used in some embodiments. Accordingly, in some embodiments, implants and components may have a shape conducive to insertion and positioning consistent with the desired approach.

Second, a plate 10 is inserted and positioned into the disc space between the first vertebral body 1 and second vertebral body 2, as shown in FIG. 5B. The plate 10 can be curvilinear in form, and can include a plurality of screw holes 20 for receiving screws to fix the plate 10 to the adjacent vertebral bodies. In some embodiments, one or more screw holes can include a longitudinal axis that differs from the longitudinal axis of one or more other screw holes, such that screws can be inserted at various angles through the plate 10. The plate 10 can also include a central screw hole 30 for receiving a cover screw 200. The plate 10 can be positioned adjacent the intervertebral spacer 5. In some embodiments, the plate 10 will contact the intervertebral spacer 5, while in other embodiments, the plate 10 will not contact the intervertebral spacer 5. Advantageously, as the plate 10 need not contact the intervertebral spacer 5, the improved plate 10 can be used with a multitude of spacers, and are not limited to use with a particular design as some conventional plates. In some embodiments, the plate 10 will be substantially or completely recessed within the disc space such that it is not exposed beyond the outer surfaces of vertebral body 1 or vertebral body 2. The plate 10 can serve to prevent or inhibit the intervertebral spacer 5 from unintentionally backing out from the disc space.

Third, screws 60 and 61 can be provided and inserted into the plate 10 to secure the plate 10 to the adjacent vertebral bodies, as shown in FIG. 5C. The screws 60 and 61 can be delivered proximate to the plate 10 and can be inserted into the plurality of screw holes 20. Depending on the configuration of the screw holes 20, the screws 60 and 61 can be inserted either at an upward or downward angle into the screw holes. As shown in FIG. 5C, two screws 60 can be inserted at an upward angle through the plate 10 and into vertebral body 1, while two screws 61 can be inserted at a downward angle through the plate and into vertebral body 2. In some embodiments, upon insertion of the screws 60 and 61 into the screw holes 20, no portion of the screws (e.g., a head) will be exposed beyond the interior surfaces of the screw holes 20, thereby reducing the risk of damage to tissue by the screws themselves. The screws 60 and 61 can help secure the plate 10 to the adjacent vertebral bodies, thereby further assisting in preventing back out of the intervertebral spacer 5.

Fourth, a cover element 110 can be provided and attached to the plate 10, as shown in FIG. 5D. The cover element 110 can include surfaces that are designed to complement the upper surface of the plate 10, such that upon compression, the cover element 110 rests securely with the plate 10. In some embodiments, the cover element 110 can be curved similar to the plate 10, and can include a surface that substantially covers the upper surface of the plate 10. To secure the cover element 110 to the plate 10, the cover element 110 can include a cover hole 112 for receiving a cover screw 200 to secure the components together. The cover hole 112 can be formed through a top surface of the cover element 110, which can be substantially or completely smooth. In some embodiments, like the plate 10, the cover element 110 will be substantially or completely recessed within the disc space such that it is not exposed beyond the outer surfaces of vertebral body 1 or vertebral body 2. This separation space serves as a buffer to reduce the risk of the cover element 110 inadvertently extending beyond the exposed outer surfaces of the adjacent vertebral bodies and causing tissue damage. The cover element 110 can function to limit unintentional back out of the plate 10 and/or its inserted screws 60 and 61, thereby reducing the risk of damage to tissue caused by back out of these components. Furthermore, the smooth upper surface of the cover element 110 helps to mitigate the risk of damage to tissue that contacts the recessed cover element 110. Once the cover element 110 is in plate with the plate 10, a cover screw 200 can be inserted through the cover element 110 and plate 10 to secure the two components together.

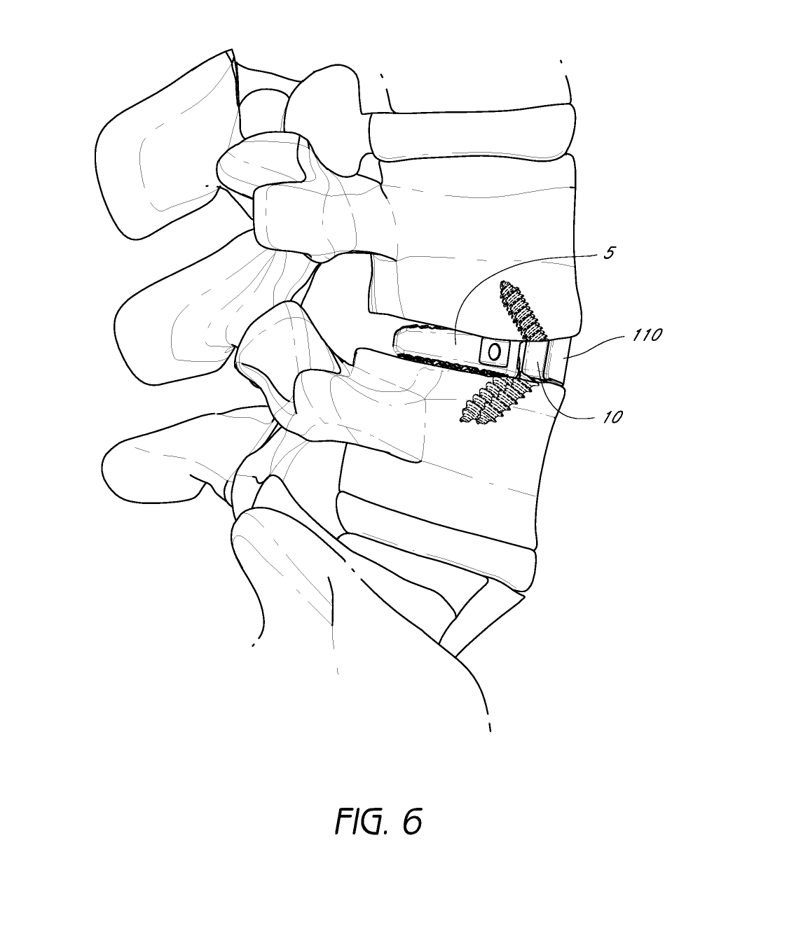

FIG. 6 illustrates a side view of an improved intervertebral plate system comprising an intervertebral spacer 5, a plate 10 and a cover element 110 that is completely recessed within an intervertebral space. Advantageously, the entire plate system is lodged and recessed within the intervertebral space, such that the risk of damage to outside tissue is minimal. Furthermore, the plate system is recessed such that the smooth top surface of the cover element 110 is exposed, which further reduces the risk of damage to tissue.

### Installation Block and Insertion Tool

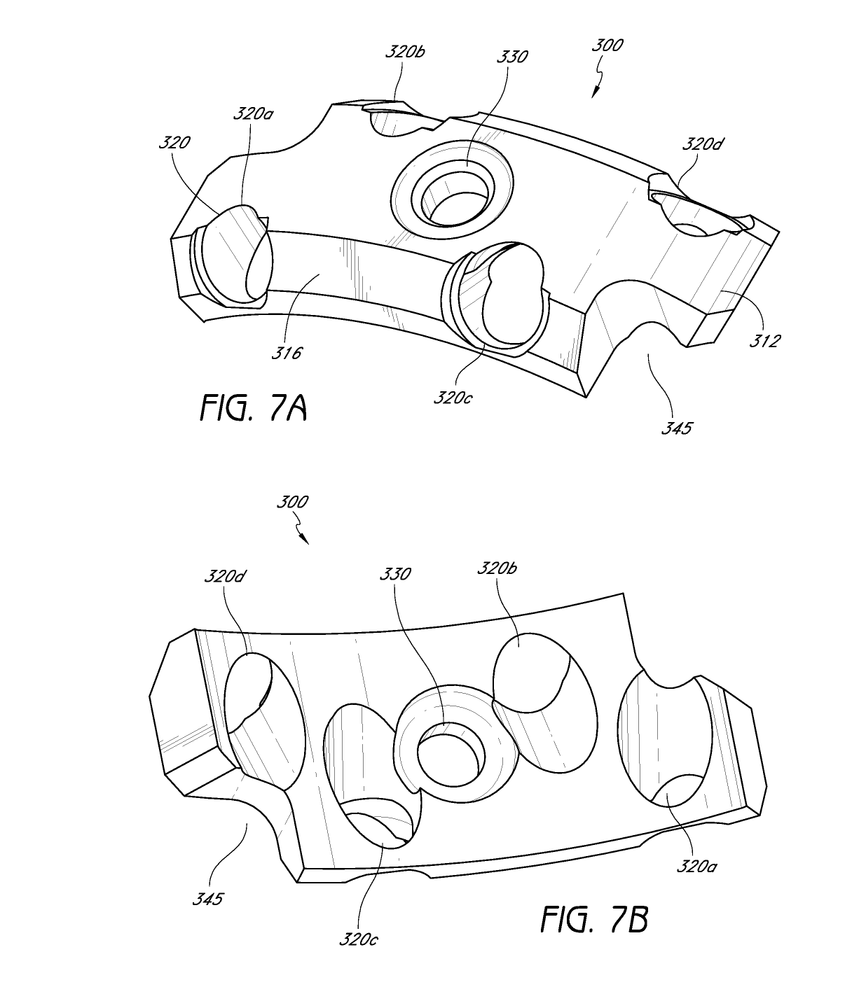

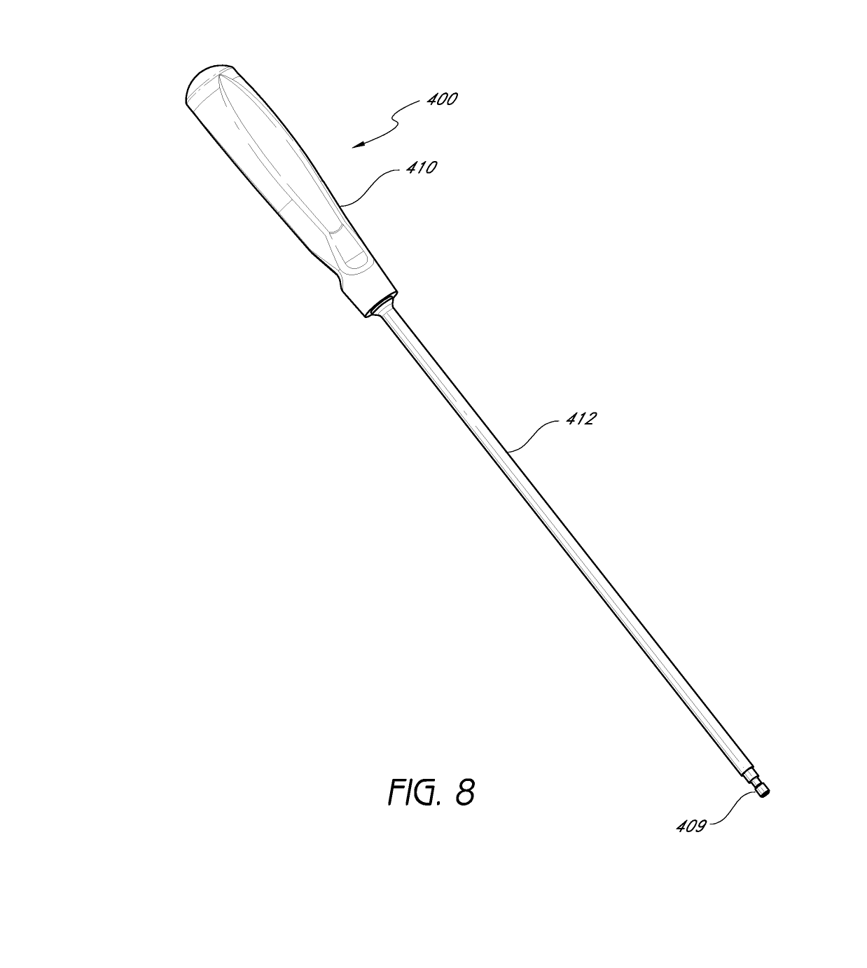

In some embodiments, the intervertebral plate system can further include an installation block shown in FIGS. 7A and 7B, and an insertion tool shown in FIG. 8. The installation block 200 advantageously helps to insert and position the plate 10 into a disc space in some embodiments. One skilled in the art will appreciate that the installation block and insertion tool may be optional in some embodiments.

As shown in FIGS. 7A and 7B, the installation block 200 comprises a top surface 312 and sidewalls 316, along with apertures 320, a block center hole 330 and corner windows 345. The apertures 320, each of which are designated respectively 320a, 320b, 320c and 320d, are angled and correspond with the plate holes 20a, 20b, 20c and 20d. Likewise, the block center hole 330 corresponds with the central screw hole 30. In some embodiments, the installation block 200 is curvilinear, and can have a substantially same curvature as the plate 10.

In some embodiments, to assist in the positioning of the plate 10 into a disc space, the plate 10 can be placed under the bottom of the installation block 300 (shown in FIG. 7B). The installation block 300 and underlying plate 10 can rest on a distal portion 409 of the insertion tool 400 (shown in FIG. 8), and can be delivered simultaneously into the disc space. In some embodiments, the installation block 300 can be slightly larger in width or other dimension than the plate 10. This additional width advantageously allows the insertion block 300 to serve as a stop that makes contact with one or more vertebral bodies, thereby ensuring proper depth and positioning of the plate 10 within the disc space. As many of the features of the installation block 300 correspond with plate 10 features, the installation block 300 also advantageously allows for proper alignment of the plate 10 within the intervertebral disc space. Once the plate 10 and/or installation block 300 are positioned within the intervertebral space, screws can be inserted into the apertures 320 of the installation block 300 and further into the holes 20 of the plate 10 to thereby fix the properly aligned plate 20 with one or more vertebral bodies.

To assist in the alignment of the plate 10 in a disc space when using the installation block 300, the installation block 300 can include one or more windows 345, as shown in FIG. 7A. In some embodiments, the windows 345 can be formed in any corner of the installation block 300. The windows 345 can be formed in any or all of the top surface 312, sidewalls 316 or bottom surface of the installation block 300. The windows 345 advantageously provide an exposed opening to an underlying plate 10, such that a surgeon can view the positioning of the plate even when recessed in a disc space.

FIG. 8 illustrates an insertion tool according to embodiments of the present application. The insertion tool 400 includes a handle 410, a shaft 412 and a distal portion 409 that can work in conjunction with the installation block 300 to deliver a plate 10 to a desirable depth and location in a disc space. In some embodiments, the distal portion 409 of the insertion tool 400 includes a groove or other element on which the insertion block 300 and plate 10 can be positioned during delivery into a disc space.

It will be apparent to those skilled in the art that various modifications and variations can be made in the present embodiments without departing from the scope or spirit of the advantages of the present application. Thus, it is intended that the present application cover the modifications and variations of these embodiments and their equivalents.

What is claimed is:

1. A spinal implant method comprising:
positioning an intervertebral spacer in an intervertebral space between two vertebrae of a patient;
positioning a plate at least partially in said intervertebral space and adjacent to the spacer, to at least inhibit the intervertebral spacer from backing out from said space;
positioning a plurality of bone screws through the plate to anchor the plate between two vertebrae of a patient; and
positioning a cover element at least partially in said intervertebral space and over the plate to inhibit the plurality of bone screws from backing out of the plate, wherein the cover element has a surface that covers a substantial portion of an upper surface of the plate and wherein the plate and cover element are substantially recessed between two vertebrae of a patient when implanted such that they do not extend significantly beyond the outer surfaces of the two vertebrae.

2. The spinal implant method of claim 1, further comprising inserting a plurality of screws into the plate to anchor the plate to the two vertebrae.

3. The spinal implant method of claim 1, further comprising inserting a cover screw through the cover element and plate to secure the cover element to the plate.

4. The spinal implant method of claim 1, wherein positioning a plate at least partially in said intervertebral space comprises substantially or completely recessing the plate in said intervertebral space.

5. The spinal implant method of claim 1, wherein positioning a cover element at least partially in said intervertebral space comprises substantially or completely recessing the plate in said intervertebral space.

6. The spinal implant method of claim 1, wherein positioning an intervertebral spacer in an intervertebral space comprises using an anterior approach.

7. The spinal implant method of claim 1, wherein positioning an intervertebral spacer in an intervertebral space comprises using a lateral approach.

8. The spinal implant method of claim 1, wherein positioning an intervertebral spacer in an intervertebral space comprises using a posterior approach.

9. The spinal implant method of claim 1, wherein positioning an intervertebral spacer in an intervertebral space comprises using a posterior-lateral approach.

10. The spinal implant method of claim 1, wherein positioning the plate at least partially in said intervertebral space and adjacent to the spacer is performed with an installation block.
